(19) United States
(12) Patent Application Publication
(10) Pub. No.: US 2018/0021054 A1
(43) Pub. Date: Jan. 25, 2018

(54) SYSTEMS AND METHODS FOR CROSSING AND TREATING AN OCCLUSION

(71) Applicant: XableCath Inc., Salt Lake City, UT (US)

(72) Inventors: Lisa K. Jordan, Salt Lake City, UT (US); Enora S. Rogers, Salt Lake City, UT (US)

(21) Appl. No.: 15/551,812

(22) PCT Filed: Feb. 16, 2016
(86) PCT No.: PCT/US16/18125

§ 371 Date: Aug. 17, 2017

Related U.S. Application Data
(60) Provisional application No. 61/117,890, filed on Nov. 25, 2008.

(51) Int. Cl. A61B 17/3207; A61M 25/00

(52) U.S. Cl. CPC A61B 17/320758; A61M 25/005; A61B 2017/22038

(57) ABSTRACT

A system is provided for crossing an occlusion within a blood vessel or cylindrical body cavity. The system includes a guide catheter and a catheter device. The guide catheter comprises a proximal portion to be disposed outside a patient and a distal portion to be disposed inside the patient adjacent to an occlusion. The system has an expandable member disposed adjacent to a distal end thereof. The expandable member can be coupled with the guide catheter or the catheter device, or an expandable member can be coupled with each of the guide catheter and the catheter device. The catheter device, which can be configured for treating an occlusion, includes a proximal portion, a distal portion, or an elongate body extending between the proximal and distal portions.

### BACKGROUND OF THE INVENTION

### Field of the Invention

This application is directed to systems and methods for treating occlusions, including crossing narrow passages of lumen segments or total occlusions.

### Description of the Related Art

A variety of techniques exist to de-bulk occluded vessel segments. While these techniques have met varying degrees of success, not all patients are successfully treated in this manner. Some patients with peripheral occlusions are left with few options other than amputation of the limb fed by the occluded artery. Such drastic techniques are obviously not available to patients with extensive occlusion of coronary and other critical arteries.

There are a number of products on the market that are designed for crossing CTOs. Intralumenal devices are one class of such products that in theory may reduce the dissection plane of a long occlusive lesion, protect collaterals and keep treatment options open.

### SUMMARY OF THE INVENTION

However, intraluminal products have operational challenges. For instance, the direction of movement of the device through the occlusion can be important to proper treatment. If the direction of movement is not substantially within a central zone, e.g., along the mid-line of the vessel, the pathway formed will be asymmetrically disposed within the vessel. This can lead to sub-optimal outcomes. For these reasons, there exists a need for a flexible, low-profile occlusion crossing catheter and system that is able to cross a region of vessel stenosis and establish a passageway sufficient to accommodate a balloon catheter or other interventional device. The crossing catheter can be combined with a guide catheter that provides good alignment a central zone of the blood vessel, e.g., a vessel midline, in some embodiments. The crossing catheter can be a delivery catheter in some implementations.

In one mode of operation, the crossing catheter is configured to act on an exposed face or portion of an occlusion. One mode of acting on the exposed face or portion is to rotate the catheter clockwise and/or counter-clockwise, e.g., in a reciprocating fashion. Because such rotation can induce some torque on the vessel wall, it can be useful to deploy a vessel stabilizer. A vessel stabilizer can be in the form of a balloon or other expandable member disposed on a catheter body. The vessel stabilizer can be on a guide catheter. The vessel stabilizer can be on a catheter device for supporting a wire and/or actively expanding an occlusion. The vessel stabilizer can be expanded into engagement with the vessel wall to hold the wall to reduce or minimize the length over which torque is transmitted along the vessel wall proximally of the occlusion.

In one embodiment, a system is provided for crossing an occlusion within a blood vessel or cylindrical body cavity. The system includes a guide catheter and a catheter device. The guide catheter comprises a proximal portion to be disposed outside a patient and a distal portion to be disposed inside the patient adjacent to an occlusion. The catheter device, which can be configured for treating an occlusion, includes a proximal portion, a distal portion, and an elongate body extending between the proximal and distal portions. The proximal portion can be disposed outside a patient in use. The distal portion is configured to be advanced from outside a patient to a portion of an occlusion. The portion of the occlusion can be a proximal portion, an exposed face or an exposed section of the occlusion. The elongate body has a lumen that extends therethrough. An inner diameter of the elongate body is configured for an interaction with a guidewire. The interaction can be a sliding interaction. The interaction can be a supporting interaction. The interaction can be a sliding and supporting interaction. The elongate body is sufficiently stiff to transmit torque for reciprocating action of the distal end. The reciprocating action can be against a portion of the occlusion. A rigid ring is disposed at the distal portion of the catheter device. The rigid ring having an occlusion reducing feature disposed at a distal end thereof. The system has an expandable member disposed adjacent to the distal end thereof. The expandable member is configured to be expanded into engagement with the vessel wall proximal of the occlusion to align the distal portion of the guide catheter with a. central region of the vessel segment just proximal to the occlusion.

In another embodiment, a catheter device is provided which can be configured for treating an occlusion. The catheter device includes a proximal portion, a distal portion, and an elongate body extending between the proximal and distal portions. The elongate body has a lumen that extends therethrough. An inner diameter of the elongate body is configured for an interaction with a guidewire. The interaction can be a sliding interaction. The interaction can include supporting the wire. The interaction can be a sliding and supporting interaction. The elongate body is sufficiently stiff to transmit torque for reciprocating action of the distal end. The reciprocating action can be against a portion of the occlusion. A rigid ring is disposed at the distal portion of the catheter device. The rigid ring having an occlusion reducing feature disposed at a distal end thereof. The catheter has an expandable member disposed adjacent to the distal end thereof. The expandable member can be configured to align the rigid ring with a portion of an occlusion. The expandable member can be configured to dilate an occlusion. The expandable member can be configured to align the rigid ring with a portion of an occlusion and also to dilate the occlusion.

In another embodiment a method is provided to treat a patient. In the method a guide catheter is advanced into a blood vessel and up to an occlusion. The guide catheter can be moved adjacent to a proximal face of the occlusion. The guide catheter can be moved to a location just proximal to an exposed face of the occlusion. An expandable member disposed on the distal end of the guide catheter is deployed in the vessel. The expandable member can be a center device. The expandable member can be configured to isolate a portion of the vessel from a torque. The expandable member can be configured to dilate an occlusion. A catheter device comprising an occlusion reducing feature is advanced. The catheter device can be advanced within the guide catheter. The catheter device can be advanced over a guide wire. The catheter device can be advanced within the guide catheter and over a guide wire. Access through the occlusion is expanded using the catheter device.

### BRIEF DESCRIPTION OF THE DRAWINGS

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIG. 1 illustrates schematically a near total occlusion;

FIG. 2 illustrates a challenge of alignment of a catheter device, such as a treatment device, with a vessel;

FIGS. 3 and 3A illustrates systems the can be used to provide access across an occlusion for therapy devices to enhance treatment of an occlusion; and

FIGS. 4 and 4A shows how the system of FIGS. 3 and 3A respectively can be used to provide good positioning and/or orientation and/or torque isolation of the catheter device in a vessel to provide for improved outcomes.

### DETAILED DESCRIPTION OF THE PREFERRED EMBODIMENT

Each and every feature described herein, and each and every combination of two or more of such features, is included within the scope of the present invention provided that the features included in such a combination are not mutually inconsistent.

Embodiments of the present invention are generally directed to catheter systems for crossing vascular stenosis, such as near total occlusions, components thereof, and methods use of such systems and components.

As used herein, the term “near total occlusion” refers to regions of vascular stenosis that reduce the cross-sectional area of the vessel lumen by >80%, in particular, by >90%, and in some cases by more than 95%. The term “total occlusion” means the entire vessel lumen is fully occupied by atheroma or other occlusive material preventing blood flow through the passage of the lumen.

As used herein, the term “substantially”, when used in reference to a linear dimension (e.g., length, width, thickness, distance, etc.) means within plus or minus one percent (1%) of the value of the referenced linear dimension.

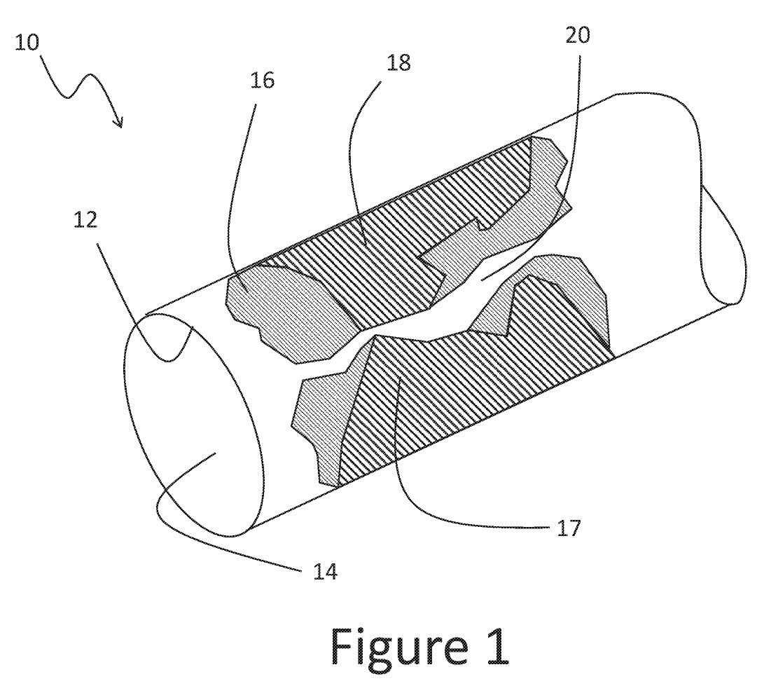

FIG. 1 illustrates a near total occlusion of a blood vessel formed by a lesion 17. The blood vessel 10 has an interior surface 12, which defines a lumen 14. In atherosclerosis, lipid and fibro muscular material accumulate in the vessel wall, forming a lesion that bulges into and occupies or occludes at least a portion of the lumen 14. Advanced-stage atherosclerotic lesions often include regions of soft plaque 16 and regions of atheroma 18. In some cases, the atheroma 18 may he calcified making access by interventional techniques difficult or impossible.

When the atheroma 18 intrudes into the lumen 14, a stenosis 20 is formed that can greatly reduce blood flow through the vessel. Angioplasty is one technique for treating a stenosis 20. In balloon angioplasty, a deflated balloon is mounted on an endovascular catheter, and the catheter is pushed along the vessel 10 until the deflated balloon occupies at least a portion of the stenosis 20. Once the deflated balloon is positioned within the stenosis 20, the balloon is inflated, pushing the atheroma 18 back toward the vessel wall and enlarging the lumen 14 within the region of stenosis 20. In some cases, an expandable stent is used to restore the lumen 14 within the region of stenosis 20.

In many cases, a guidewire is pushed ahead of the endovascular catheter to aid catheter travel through the blood vessel. The guidewire is thin and has a smaller profile than the catheter. Often, the catheter has a central lumen that accommodates the guidewire, and the catheter rides along the guidewire. This configuration of catheter is referred to as an “over-the-wire” catheter.

In some cases, the stenosis 20 is so narrow that the balloon catheter is unable to follow the guidewire through the stenosis. Rather, the balloon catheter can get hung-up or blocked at the proximal or distal end (depending on the direction of approach) of the stenosis 20. In such a case, angioplasty is precluded because it is not possible to position a deflated balloon within the stenosis 20. In some cases, the atheroma 18 forms a calcified plug that precludes passage of the guidewire through the stenosis 20.

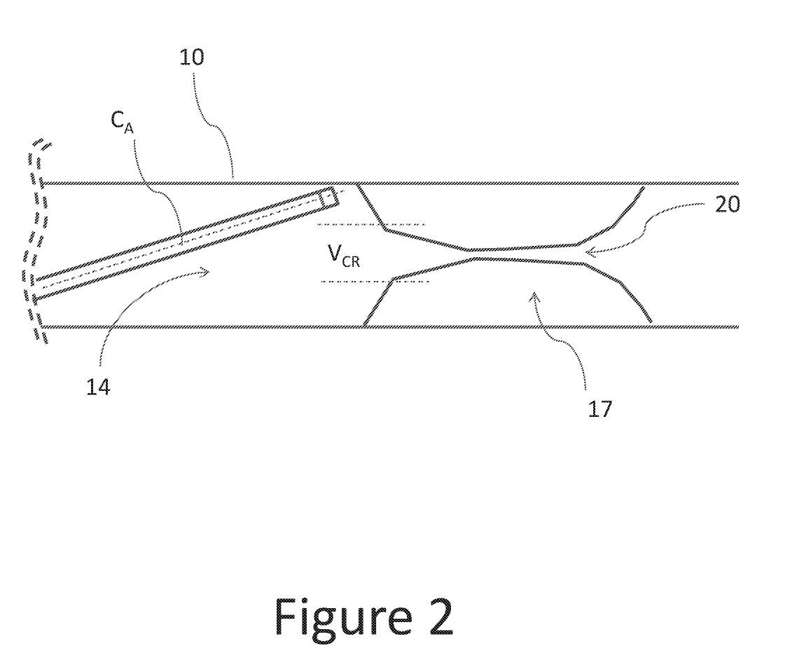

FIG. 2 shows a catheter device C disposed in the vessel 10. The catheter device C extends along a longitudinal axis C_{A} has been delivered to the region of the lesion 17. The vessel 10 is shown as having a straight trajectory. In reality, the vessel 10 may be curved but generally will have a central region V_{CR.} FIG. 2 shows that the longitudinal axis C_{A} is disposed outside the central region V_{CR}. The result is a misalignment between the longitudinal axis C_{A} and a central axis of the vessel 10. As a result, the operative end of the catheter device C when brought into contact with the lesion 17 does not act on the narrows surrounding the stenosis 20. In a worst case, the distal end of the catheter device C could act on the vessel wall. In many suboptimal cases, the distal end of the catheter device C could act to create a passage where the length of the lesion 17 is longer, resulting in a much longer procedure. In other suboptimal cases, the catheter device C may create access in offset position resulting in asymmetrical widening of the vessel. While any amount of widening can provide some clinical benefit, if one side of the vessel 10 must accommodate a greater volume of compressed lesion 17 one result could be accelerated restenosis from that side of the vessel.

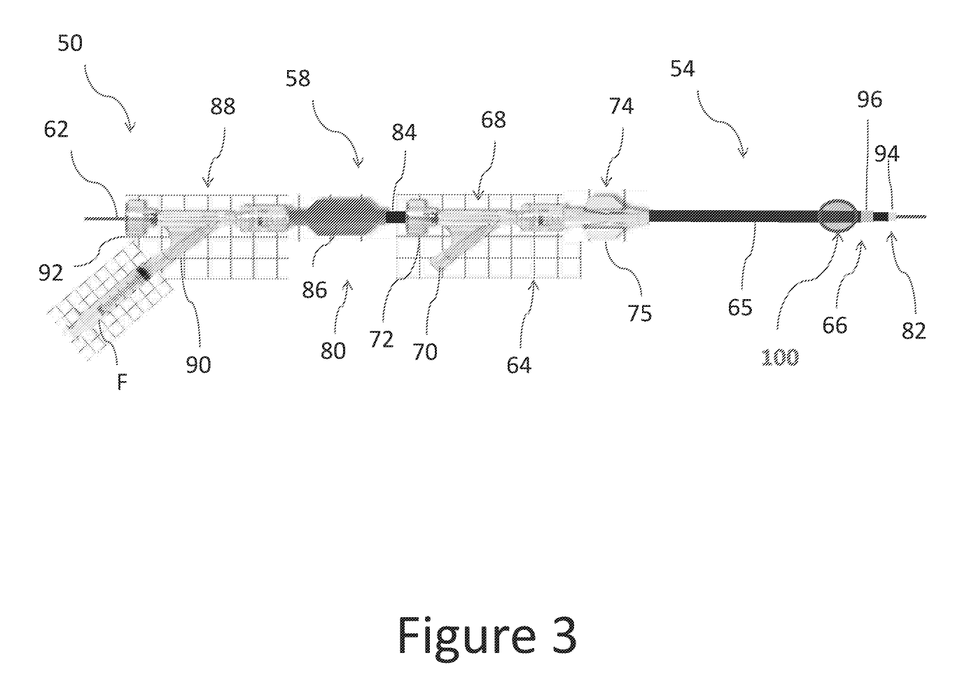

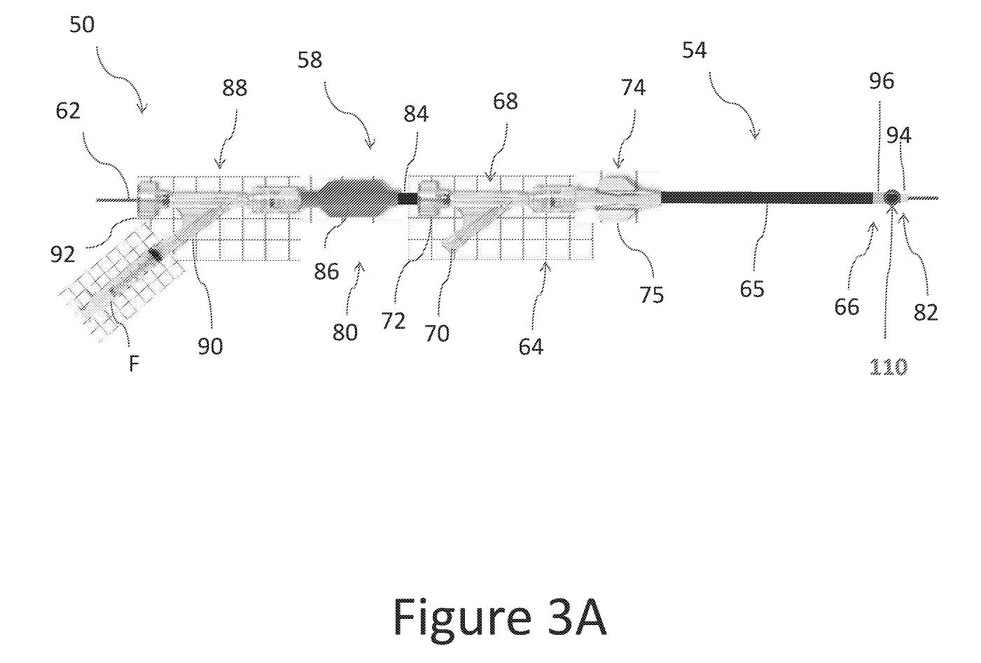

FIG. 3 illustrates an occlusion crossing system 50 that can be used to improve a clinician's ability to pass a balloon catheter or other therapy device across a blockage formed by the lesion 17. The occlusion crossing system 50 includes a sheath 54 and a catheter device 58. The catheter device 58 is provided for clearing a passage through the lesion 17 to enlarge the access therethrough, which may involve cutting the occlusion. For this reason, in some passages the catheter device 58 is referred to as a cutting catheter. The sheath 54 can be used to enclose and/or guide the catheter device 58 between a vascular access site and an occlusion. The sheath 54 thus provides protection for the un-occluded vessel(s) through which the catheter device 58 is delivered. The occlusion crossing system 50 can also include a guidewire 62 to help access or cross an occlusion.

The guidewire 62 can take any suitable form. It can be a long slender wire with no lateral protuberances or it can have one or more lateral extensions. For example a plurality of barriers or shoulders can be provided along a distal length of the guidewire 62 to engage and retain portions of the lesion 17. The guidewire 62 can have an anchor, such as a helical structure adapted to be advanced rotationally into the lesion to engage and hold it. These are examples of structures that can positively engage and hold the lesion 17. When so engaged, these structures can provide a counter traction for holding the position of the lesion while catheter device 58 (or variant herein) is advanced into the lesion to enhance access across the lesion. Examples of barriers and anchor are discussed in U.S. Pat. No. 5,443,443 and U.S. Pat. No. 5,047,040, which are hereby incorporated by reference herein in their entirety.

The sheath 54 comprises a proximal end 64, a distal end 66, and a lumen extending through an elongate body 65 disposed between the ends 64, 66. The lumen is sized to receive the catheter device 58 as discussed further below. The proximal end 64 of the sheath 54 is preferably configured to be coupled with other devices. For example, a branched access port 68 can be provided at the proximal end 64. A first branch 70 can be provided to couple with a fluid source. A second branch 72 can be aligned with the lumen of the sheath 54 to provide in-line access to the lumen of the sheath 54. One or both of the branches 70, 72 can have a valve structure to limit, minimize or eliminate blood loss. A tuohy-borst attachment can be provided on one or both of the branches 70, 72. In one embodiment, the proximal end 64 includes a modular coupling 74 that enables the branched access port 68 to be decoupled from the elongate body 65 if access via the branches is not required or for certain phases of procedures where the branches are not needed and might be in the way if not removed from the procedure zone. The coupling 74 can include torque structures 75 on opposite sides thereof.

The catheter device 58 is configured to be advanced to the occlusion 20 to provide a therapy as discussed herein. The catheter device 58 comprises a proximal end 80, a distal end 82, and a lumen extending through an elongate body 84 disposed between the ends 80, 82. The lumen is sized to provide access for a balloon catheter or other therapy device, for fluid to be injected or withdrawn, and/or for material of the occlusion 20 to be lodged. The elongate body 84 has sufficient rigidity for deliverability and for providing cutting or segmenting action at the occlusion 20. For example the body 84 can be configured to provide 1:1 torque. As discussed below, braids and coils are contemplated as structures providing pushability and flexibility for various applications, including peripheral, coronary and neuro-vascular applications.

The elongate body 84 has a length sufficient to reach a treatment site such as a peripheral, coronary, or neuro-vascular treatment site. For example, for ipsa-lateral treatment, the elongate body 84 can be between about 40 and about 100 cm, e.g., about 80 cm. For a treatment in the iliac artery, the elongate body 84 can be about 60 cm. For a treatment in the superficial femoral artery (SFA), the elongate body 84 can be between about 140 and 160 cm. For a treatment in the coronary arteries, the elongate body 84 can be between about 110 cm and about 140 cm. For neurovascular applications the elongate body 84 can be between about 130 cm and about 180 cm, e.g., about 150 cm. The sheath 54 can be about 10 cm to about 20 cm shorter than the catheter device 58. The elongate body 65 can be 10-20 cm shorter than the elongate body 84. More generally, the sheath 54 or elongate body 65 can be shorter than the catheter device 58 or elongate body 84 by an amount sufficient to provide a working length.

The length of the elongate body 84 can also be a function of the path to be traversed by the catheter device 58 to treat the patient. The access point for inserting the catheter device 58 can be in the groin on the patient, in the arm of the patient, or in the lower leg of the patient. Access at the groin can be at a femoral artery or vein. Access in the arm can be at a radial artery or vein. Access in the lower leg can be at a pedal artery or vein. Treatment site can be anywhere in the body that occlusions may form and where such occlusions provide risk to viable tissues. A catheter length of 200 cm or more can be used to traverse from the pedal artery to neurovaculature. A catheter length of 200 cm or more can be used to traverse from the radial artery to vessels below the ankle. A shorter length of approximately 150 cm can be used to reach the neurovaculature from the groin. A shorter length of approximately 110-120 cm can be used to reach the coronary vasculature from the groin. Still shorter lengths, e.g., 80-90 cm can be used to access vasculature of the foot from the pedal artery or from the groin.

For longer catheters, the elongate body 84 can be configured to facilitate access to remote vessels. For instance, the stiffness of the body can be tailored to sustain substantially 1:1 toque. A proximal zone can include a stiff structure, such as a hypotube. A zone distal the proximal zone can be more flexible. For instance a continuous change in a support member such as a braid can make the elongate body 84 progressively more flexible toward the distal end. Also, the wall thickness and or diameter of the elongate body 84 can be reduced toward the distal end. As discussed above, the tip structure, e.g., the occlusion clearing implement 94 can be much stiffer than middle regions of the elongate body 84.

FIG. 3 shows that the lumen in the body 84 can receive the guidewire 62 in certain embodiments and for certain techniques. The proximal end 80 of the catheter device 58 preferably has a handle 86 that is used to actuate the catheter 58. The handle 86 is configured to transmit a torque. The handle 86 can transmit a torque to the cutting catheter 58. The proximal end 80 can also include a branched access port 88 or other access device. A first branch 90 can be provided to couple with a fluid source F. A second branch 92 can be aligned with the lumen of the cutting catheter 58 to provide in-line access to the lumen in the body 84. One or both of the branches 90, 92 can have a valve structure to limit, minimize or eliminate blood loss. A tuohy-borst attachment can be provided on one or both of the branches 90, 92. In one embodiment, the branched access port 88 can be detached from the handle 86 when access via the branches 90, 92 is riot required or for certain phases of procedures where the branches are not needed and might be in the way if not removed from the procedure zone. In one technique, the branched access port 88 is left in place when torque* the catheter 58 because the first branch 90 provides a higher torque than the handle 86 in an optional system and technique.

The distal ends 66, 82 can be configured to be incompressible and/or radiopaque. The distal end 82 can be configured to engage and disrupt the occlusion 20 to enhance access through the stenosis 20. The distal end 82 preferably is stiffer than the elongate body 84 at locations proximal of the distal end 82. The end 82 includes an occlusion clearing implement 94, which can be one or more teeth, a continuous but abrasive surface for removing matter, a concave scooping structure for separating volumes of the matter from the occlusion 17 or other structures discussed herein. As discussed further below, the implement 94 or the system 50 are configured to follow a directed path and not to cause vessel injury in regions not being treated. The implement 94 can be radiopaque to provide visualization of the cutting catheter 58 when disposed in the vasculature.

The sheath 54 is configured to slideably and rotatably receives the catheter device 58. The inner surface of elongate body 65 and/or the outer surface of elongate body 84 can be configured to ease a retracting or extending motion in an axial direction, e.g., along the longitudinal axis of the body 65 or the body 84. Either of these surfaces can have a lubricious coating, for example. In one embodiment, the inner surface of the body 84 includes an expanded polytetrafluoroethylene (ePTFE) or other similar liner. As a result, the end 82 of the cutting catheter 58 can be pulled back into the end 66 of the sheath 54 for delivery or pushed out from the end 66 for engagement with the occlusion 20. The end 66 is configured to minimize out-of-round conditions of the sheath 54. In particular, a support ring 96 of the body 65 can be made more rigid than portions of the elongate body proximal of the distal portion 96 such that the elongate body 84 can freely rotate within the body 65. For example the support ring 96 can include a metal or ceramic cylinder that has hoop strength preventing it from being deformed when urged against an occlusion. The rigidity of the support ring 96 provides the advantage that the distal end 66 will maintain its pre-delivery configuration or will be deformed only by an amount that would not restrict rotation of the body 84 and thereby the end 82. The support ring 96 can be made of a radiopaque material to enhance visibility of the sheath 94 and the system 50.

FIG. 3 also shows that the system 50 can include an alignment feature or structure. In one embodiment, an expandable member 100 is provided on the sheath 54. The expandable member 100 is configured to be expanded into engagement with a vessel wall. In one embodiment, the expandable member 100 is a balloon that is in fluid communication with an inflation lumen formed through the elongate body 65. The lumen can be in fluid communication with a source of inflation media coupled with the branch 70. FIG. 3 shows the expandable member 100 in an expanded state. The expandable member 100 can be collapsed to be in a low profile. When collapsed, the distal end 66 and the expandable member 100 can be advanced into a blood vessel and situated near an occlusion.

In one variation, the expandable member 100 does not require an inflation medium under pressure. For example, the expandable member 100 can be a self-expanding member. The expandable member 100 can include a self-expanding member that can be selectively deployed inside a vessel adjacent to an occlusion. In one self-expanding member, a highly elastic member is provided. The highly elastic member can include a material such as nitinol that is able to store sufficient strain energy to be collapsed for low profile delivery but to expand into engagement with a vessel wall. Deployment can be caused by pulling back on a sheath or other restraining device. In another embodiment, the expandable member 100 can be formed of a heat actuated shape memory material. In such embodiments, the heat of the blood can be used to trigger expansion from a low profile for delivery to a higher profile for engaging the vessel.

Methods of using the occlusion crossing system 50 or similar systems with any of the alternative components described herein are discussed below and in PCT Application No. PCT/US2014/056162, filed Sep. 17, 2014, which is incorporated by reference herein in its entirety.

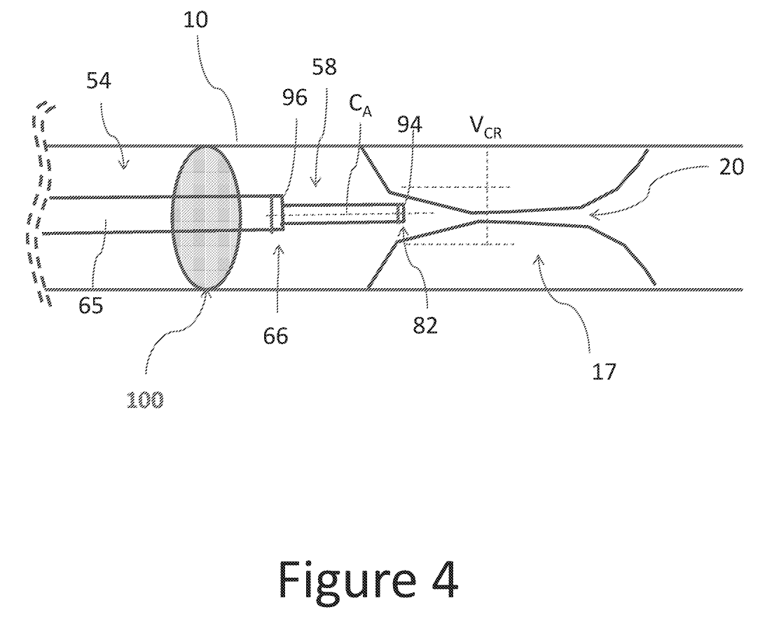

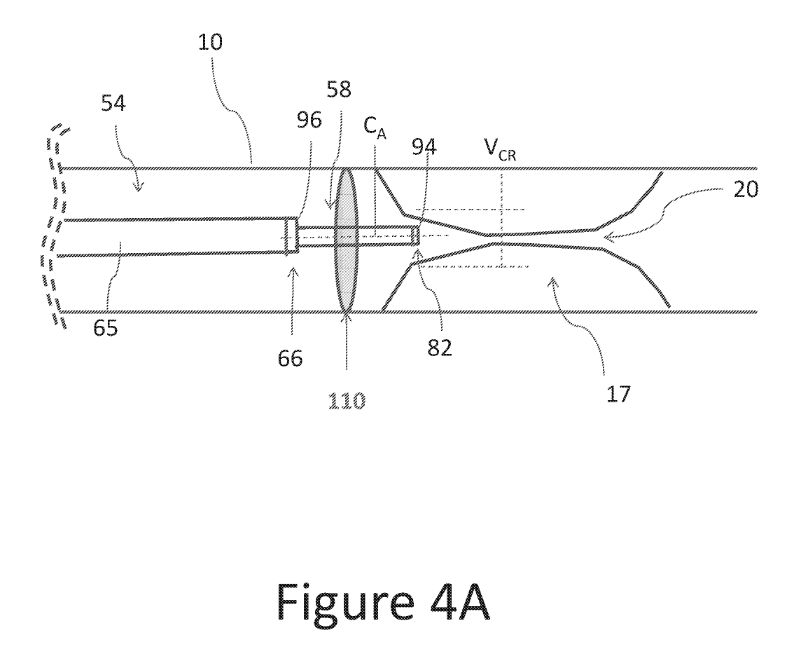

FIG. 4 illustrates a method of treating a patient. Prior to the portion shown in FIG. 4, the sheath 54 is advanced into a percutaneous access site. The distal end 66 is advanced through the vasculature to a location near an occlusion 20. The sheath 54 can carry the catheter device 58 with it the treatment site. In certain methods, the sheath 54 is delivered first and after situated in the vasculature, allows the catheter device 58 to he delivered within the lumen of the sheath 54 to the treatment site. In at least this sense, the sheath 54 acts as a guide catheter. The guide catheter 54 can be moved adjacent to a proximal face of the occlusion, as shown in FIG. 4.

The guide catheter 54 can be moved to a location just proximal to an exposed face of the occlusion. A centering device disposed on the distal end of the guide catheter 54 is deployed in the vessel. The center device can include the expandable member 100, e.g., a balloon or self-expanding member.

The catheter device 58 can then be advanced out of the distal end 66 of the guide catheter 54. FIG. 4 shows that an occlusion reducing feature 94 disposed at the distal end 82 of the catheter device 58 can be advanced toward the occlusion 20. The longitudinal axis C_{A} of the catheter device 58 is retained in a central region V_{CR} of the vessel 10. Because the narrow passage defined through the occlusion tends to be in the central region V_{CR} the occlusion reducing feature 94 is guided to region likely to have the shortest path through the occlusion 20. The shortest path is likely to provide the shortest procedure time, which is an advantage for patient recovery. Also, the material that forms adjacent to the narrows of the occlusion 20 is likely to be most recently formed. As such, it may be the softest and easiest to cross. By aligning the catheter device 58 and/or the occlusion reducing feature 94, there is a focalizing effect of the therapy on the portion of the occlusion that can be crossed in a most efficient manner. This also can contribute to shorter procedure times. Shorter procedures are also cost saving for the health care system if outcomes are still strong.

Though not shown in FIG. 4, the method can include some guidance over the guidewire 62. In certain embodiments, reciprocation of the catheter device 58 and advancement of a wire 62 can be coordinated to expedite passage through the occlusion 20. Access through the occlusion 20 is expanded using the catheter device 58.

in some embodiments, the elongate body 84 of the catheter device 58 is hollow and cylindrical or substantially cylindrical, having an inner diameter between about 0.94 mm to about 1.07 mm. In several embodiments, the elongate body 84 has an outer diameter between about 1.12 mm to about 1.37 mm. In some embodiments, the central lumen of the elongate body 84 is configured to accommodate a guidewire 62, as mentioned above. In at least one embodiment, the inner diameter of the elongate body 84 is less than 10% larger than the outer diameter of the guidewire 62. In other embodiments, a smaller gap on a percentage basis may be provided. For example, some embodiments provide a less than 5% gap between the inner diameter thereof and an outer diameter of a guidewire 62.

In other embodiments and techniques, the guidewire 62 is used to track the catheter device 58 and specifically the occlusion reducing feature 94 to the stenosis. Once in position, the guidewire 62 could be withdrawn and the occlusion reducing feature 94 can be used to enhance access across the occlusion. If the guidewire 62 is in place the occlusion reducing feature 94 may rotate about the outer surface of the guidewire 62 independently either exposed in the vessel or in the sheath 54. Thus, in some embodiments, the guidewire 62 is not required to be in place or to rotate with the system for the device to function. In other embodiments and for certain applications, a guidewire 62 may not be used even for delivery of the system. For example, if the vessel segment is straight there may not be a need for a guidewire 62. In such cases, the occlusion reducing feature 94 preferably is configured to enhance access across an occlusion without support from a guidewire 62.

In some variants, the occlusion reducing feature 94 is configured to remove lesion tissue through different modes of operation including cutting, tearing, shaving, or abrading the lesion tissue. The occlusion reducing feature 94 may be configured to use one, or more than one, method of removing lesion tissue. In several embodiments, the occlusion reducing feature 94 provides lateral support to the guidewire 62 as the guidewire 62 is advanced through a stenosis. In some embodiments, the occlusion reducing feature 94 can prevent the guidewire 62 from buckling as the guidewire 62 is advanced through an occlusion or a near total occlusion. In providing this function, the occlusion reducing feature 94 can be configured with a bore having a diameter that is close to that of the guidewire 62, e.g., within about 10% of the diameter of the guidewire 62. The gap between the guidewire 62 and the occlusion reducing feature 94 should be large enough to keep resistance to relative movement (advancement and/or rotation) between these components to an acceptable level for tracking and twisting. In addition, the occlusion reducing feature 94 may be used as an exchange device for changing guidewires or other interventional devices without losing position or access to the target lesion. The lumen in the occlusion reducing feature 94 can be used for drug delivery and contrast injection as needed.

One feature that aids in guidance of the cutting catheter 58 whether guided by a wire or a guide catheter is the configuration of a rigid distal portion, for example of the occlusion reducing feature 94. The occlusion reducing feature 94 can be configured to minimize wandering within a blood vessel. In particular, blood is subject to varying pressures and certain peripheral blood vessels have a relatively high mobility. By making the length of the occlusion reducing feature 94 greater than the inner diameter of the occlusion reducing feature 94, the occlusion reducing feature 94 tends to remain generally straight in the vessel. In some embodiments, the length of the occlusion reducing feature 94 is more than two times the diameter of the tip. In some embodiments, the length of the occlusion reducing feature 94 is more than two and one-half times the diameter of occlusion reducing feature 94. In some embodiments, the length of the occlusion reducing feature 94 is more than three times the diameter of the occlusion reducing feature 94, The length of the occlusion reducing feature 94 can be from 1-5 times the diameter of the occlusion reducing feature 94 in certain embodiments.

Other methods can be performed by virtue of the expandable member 100 being present in the procedure zone. For example, the expandable member 100 can be configured to expand to engage the vessel 10 proximal of the occlusion. Thereafter, the catheter device 58 can be rotated in the vessel 10 to cause the occlusion to be removed. Such rotation may transmit torque to the vessel wall. But, the expandable member 100 can minimize or reduce the propagation of such torque upstream of the occlusion 20 to locations that are also upstream of the expandable member 100. In one variation, the expandable member 100 can be un-expanded (e.g., deflated) to permit blood to enter the procedure zone between the expandable member 100 and the occlusion 20. This can provide a form of natural irrigation of the zone. The expandable member 100 may be un-expanded when the catheter device 58 is being held stationary, such as when access across the occlusion 20 is being expanded by urging a wire distally within the catheter device 58. Thereafter, the expandable member 100 can once again be expanded to facilitate the rotation of the catheter device 58 as described herein.

In sonic embodiments, one or more of [a] compression force or torsion to the guidewire 62 or [b] compression force or torsion is applied to the elongate body 84 to expand or create an access path through the occlusion. The occlusion reducing feature 94 can be used to gently abrade the lesion 17. Once the occlusion reducing feature 94 makes contact with the lesion 17, a user applies torque to the handle 86, causing the handle 86 to rotate about the guidewire 62. The elongate body 84 transmits the torque to the occlusion reducing feature 94, causing the occlusion reducing feature 94 to slide over the surface of the lesion 17. In many embodiments, a user rotates the handle 86 in alternating clockwise and counterclockwise directions. In some embodiments, the handle 86 is rotated in only one direction. In some embodiments, a user applies compressive forces by pushing the handle 86 in the distal direction. In some embodiments, a user applies simultaneously compressive and torsional forces by pushing the handle 86 in the distal direction while rotating handle 86 about the guidewire 62.

Other methods can incorporate some dilatation of the occlusion using the expandable member 100. For example the expandable member 100 can initially be expanded proximally of the occlusion 20. Thereafter, the catheter device 58 can expand access across the occlusion 20 sufficiently to enable the expandable member 100 to be urged into the occlusion 20. The expandable member 100 can then be expanded to further enlarge access across the occlusion 20. This method can be iterated by incrementally moving the catheter device 58 and then the expandable member 100 farther and farther into and across the occlusion 20.

In a further variation, the sheath 54 can be configured to remove particles from the procedure zone. For example, suction can be applied to the first branch 70 of the access port 68 to draw matter liberated from the occlusion 20 out of the patient's body. If the expandable member 100 is expanded prior to action of the catheter device 58 the liberated matter maybe substantially retained in the space between the expandable member 100 and the occlusion 20. By aspirating during rotation of the catheter device 58 at least until after the catheter device 58 stops rotating, the propagation of particles downstream can be reduced or minimized.

FIGS. 3A and 4A show further variations in which the catheter device 58 has an expandable member 110 disposed on a distal end thereof. The expandable member 110 can serve many of the same functions as the expandable member 100. The expandable member 110 can expand to move the longitudinal axis C_{A} of the catheter device 58 toward the central region V_{CR} of the vessel 10. This can provide some of the same advantages discussed above, e.g., positioning the occlusion clearing implement 94 at the location corresponding to the fastest procedure, as discussed above. This provides the further advantage of enabling the sheath 54 to be simplified or eliminated. For small vessels, eliminating the sheath 54 may be the difference between being able to treat and not being able to treat using these catheter techniques. In one method, the expandable member 110 is a balloon that can be inflated to high pressure to facilitate a dilatation of the vessel 10. For example, the balloon can be configured to be overinflated by as much as 20% in one mode. The expandable member 110 can be a balloon that is also configured to be inflated to a lesser extent to permit the catheter device 58 to be rotated within a vessel. In other words, the expandable member 110 can be expanded to loosely, slideably engage the wall of the vessel 10 without injuring it but at the same time placing the C_{A} of the catheter device 58 adjacent to or within the central region V_{CR}.

The above presents a description of modes contemplated of carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above which are fully equivalent. Consequently, it is not the intention to limit this invention to the particular embodiments disclosed. On the contrary, the intention is to cover modifications and alternate constructions coming within the spirit and scope of the invention as generally expressed by the following claims, which particularly point out and distinctly claim the subject matter of the invention.

What is claimed is:

1. A system for crossing an occlusion within a blood vessel or cylindrical body cavity, comprising:
a wide catheter comprising a proximal portion to be disposed outside a patient and a distal portion to be disposed inside the patient adjacent to an occlusion;
a catheter device, comprising:
a proximal portion to be disposed outside a patient in use;
a distal portion to be advanced from outside a patient to a portion of an occlusion;
an elongate body extending between the proximal portion and the distal portion, the elongate body having a lumen extending therethrough with an inner diameter configured for an interaction with a guidewire, the elongate body being sufficiently stiff to transmit torque for reciprocating action of the distal end; and
a rigid ring disposed at the distal portion, the rigid ring having an occlusion reducing feature disposed at a distal end thereof; and
an expandable member disposed adjacent to a distal end of the system;
wherein the expandable member is configured to be expanded into engagement with the vessel wall proximal of the occlusion to align the distal portion of the catheter device with a central region of the vessel segment just proximal to the occlusion.

2. The system of claim 1, wherein the expandable member comprises an inflatable balloon.

3. The system of claim 1, wherein the expandable member comprises a self-expanding member.

4. The system of claim 1, wherein when the expandable member is in an expanded configuration against the vessel wall, a trajectory of the catheter device is aligned with the trajectory of the vessel just proximal to the occlusion.

5. The system of claim 1, wherein the expandable member is disposed on the guide catheter.

6. The system of claim 1, wherein the expandable member is disposed on the catheter device.

7. A method, comprising:
advancing a guide catheter into a blood vessel and adjacent to an occlusion;
deploying an expandable member disposed on the distal end of the guide catheter;
advancing a catheter device comprising an occlusion reducing feature disposed therein; and
expanding access through the occlusion using the catheter device.

8. The method of claim 7, wherein deploying comprising expanding a balloon on the distal end of the guide catheter.

9. The method of claim 7, wherein expanding access across the occlusion comprises rotating the catheter device in a reciprocating fashion to abrade the occlusion.

10. The method of claim 7, wherein expanding access across the occlusion comprises rotating the catheter device in a reciprocating fashion to cut the occlusion.

11. The method of claim 7, wherein expanding access across the occlusion comprises alternately supporting distal advancement of a wire through the catheter device and rotating the catheter device in a reciprocating fashion to separate matter from the occlusion.

12. The method of claim 7, further comprising collecting matter separated from the occlusion in the catheter or guide catheter.
